(19) United States
(12) Patent Application Publication
(10) Pub. No.: US 2009/0177111 A1
(43) Pub. Date: Jul. 9, 2009

(54) System and method for displaying contact between a catheter and tissue

(76) Inventors: Stephan P. Miller, Minneapolis, MN (US); Don C. Deno, Andover, MN (US); Lewis C. Hill, II, Roseville, MN (US); Yuriy Malinin, Edina, MN (US); Holly Cotner, St. Paul, MN (US); Suzann R. Mouw, White Bear Lake, MN (US); Patrick W. Drigans, Buffalo, MN (US); Saurav Paul, Minnetonka, MN (US)

Correspondence Address: SJM/AFD-WILEY, 14901 DEVEAU PLACE, MINNETONKA, MN, 55345-2126, US

(21) Appl. No.: 12/353,897

(22) Filed: Jan. 14, 2009

Related U.S. Application Data
(63) Continuation-in-part of application No. 12/253,637, filed on Oct. 17, 2008.
Application No. 12/253,637 is a continuation-in-part of application No. 12/095,688.
(60) Provisional application No. 61/020,806, filed on Jan. 14, 2008.

(51) Int. Cl. A61B 5/053; A61B 5/00

(52) U.S. Cl. 600/547; 600/587

(57) ABSTRACT

A system and method for assessing and displaying a degree of contact between a sensor, an electrode, and tissue in a body is provided. Values for the sensor are read, and a degree of contact is calculated. This degree of contact is displayed to a clinician in a variety of ways to indicate the degree of contact to the clinician. The system and method find particular application in ablation of tissue by permitting a clinician to create lesions in the tissue more effectively and safely.

This application is a continuation-in-part of U.S. provisional application No. 61/020,806, filed Jan. 14, 2008. This application is also a continuation-in-part of U.S. application Ser. No. 12/253,637, filed Oct. 17, 2008, which is a continuation-in-part of U.S. application Ser. No. 12/095,688 filed May 30, 2008. U.S. application Ser. No. 12/095,688 is a national stage application of, and claims priority to, International Application No. PCT/US2006/061714, filed Dec. 6, 2006. The International Application was published in the English language on Jun. 14, 2007 as International Publication No. WO 2007/067941 A2 and itself claims the benefit of U.S. provisional application No. 60/748,234, filed Dec. 6, 2005. All of the foregoing are hereby incorporated herein by reference as though fully set forth herein.

### BACKGROUND OF THE INVENTION

a. Field of the Invention

This invention relates to a system and method for displaying a degree of contact or coupling between a portion of a catheter, an electrode on a catheter, and tissue in a body. In particular, the instant invention relates to a system and method for displaying the degree of contact between electrodes on a diagnostic and/or therapeutic medical device such as a mapping or ablation catheter and tissue, such as cardiac tissue.

b. Background Art

Catheters with various types of sensors and active elements are used on a variety of diagnostic and/or therapeutic medical procedures. For example, electrodes may be used on cardiac mapping catheters to determine electric potentials in the heart. Likewise, catheters with electrodes or magnetic coils are used to generate an image of the internal geometry of a heart, and may be used (separately or in combination) to match the electrical potentials with a location on the tissue. Electrodes and other active elements are also used on ablation catheters to create tissue necrosis in cardiac tissue to correct conditions such as atrial arrhythmia (including, but not limited to, ectopic atrial tachycardia, atrial fibrillation, and atrial flutter). Arrhythmia can create a variety of dangerous conditions including irregular heart rates, loss of synchronous atrioventricular contractions, and stasis of blood flow, which can lead to a variety of ailments and even death. It is believed that the primary cause of atrial arrhythmia is stray electrical signals within the left or right atrium of the heart. The ablation catheter imparts ablative energy (radiofrequency energy, cryoablation, lasers, chemicals, high-intensity focused ultrasound, etc.) to cardiac tissue to create a lesion in the cardiac tissue. This lesion disrupts undesirable electrical pathways and thereby limits or prevents stray electrical signals that lead to arrhythmias.

The safety and effectiveness of many of diagnostic and/or therapeutic devices is often determined in part by the proximity of the device to the target tissue. Mapping catheters often use electrodes to map the location of the target tissue. In these applications, the distance between the electrodes and the target tissue affects the strength of the electrical signal and the identity of the mapping location. The safety and effectiveness of ablation lesions is determined in part by the proximity of the ablation element to target tissue and the effective application of energy to that tissue. If the ablation element is positioned improperly, too far from the tissue or has insufficient contact with the tissue, the lesions created may not be effective. On the other hand, if a catheter contacts the tissue with excessive force, the catheter may perforate or otherwise damage the tissue (by overheating). It is therefore beneficial to assess whether or not a catheter is in contact with the tissue, and if so, the degree of contact between the catheter and the tissue.

Contact between a catheter and tissue can be determined by numerous methods with varying success. Some methods are subjective and difficult to quantify. Others known in the art are typically reported to the end user as a binary contact/no contact result, or as a simple number.

For example, contact has been determined using clinician sense, fluoroscopic imaging, intracardiac echo (ICE), atrial electrograms (typically bipolar D-2), pacing thresholds, evaluation of lesion size at necropsy and measurement of temperature change at the energy delivery site.

Although a clinician can evaluate contact based on tactile feedback from the catheter and prior experience, the degree of contact is difficult to quantify as the measurement depends largely on the experience of the clinician and is also subject to change based on variations in the mechanical properties of catheters used by the clinician. The determination is particularly difficult when using catheters that are relatively long (such as those used to enter the left atrium of the heart).

Because fluoroscopic images are two-dimensional projections and blood and myocardium attenuate x-rays similarly, it is difficult to quantify the degree of contact and to detect when the catheter tip is not in contact with the tissue. Fluoroscopic imaging also exposes the patient and clinician to radiation.

Intracardiac echo is time consuming and it is also difficult to align the echo beam with the ablation catheter. Further, intracardiac echo does not always permit the clinician to confidently assess the degree of contact and can generate unacceptable levels of false positives and false negatives in assessing whether the electrode is in contact with tissue.

Atrial electrograms do not always correlate well to tissue contact and are also prone to false negatives and positives. Pacing thresholds also do not always correlate well with tissue contact and pacing thresholds are time-consuming and also prone to false positives and false negatives because tissue excitability may vary in hearts with arrhythmia. Evaluating lesion size at necropsy is seldom available in human subjects, provides limited information (few data points) and, further, it is often difficult to evaluate the depth and volume of lesions in the left and right atria. Finally, temperature measurements provide limited information (few data points) and are difficult to evaluate in the case of irrigated catheters.

Another method of assessing contact between the catheter electrode and tissue is the use of force sensors incorporated into the catheter to measure contact force between the catheter tip and tissue. In addition, recent methods go beyond physical contact and measure the degree to which a catheter is electrically coupled to the tissue. Particularly for radio-frequency (RF) ablation catheters, a measure of electrical coupling may be more relevant to ablation safety and efficacy in different types of tissue and in different types of catheter tip to tissue surface alignment (perpendicular versus parallel orientation).

While numerous methods of evaluating contact are known, the inventors herein have recognized a need for a system and method for determining a degree of contact between a catheter and tissue and providing the clinician with a clinically useful display of that degree of contact. In particular, the rapid pace of modern catheter procedures already places tremendous demands on the clinician to mentally analyze and track the location and actions of the catheter. Providing the clinician with a system that readily and clearly quantifies the degree of contact between the catheter and the tissue will free up the clinician's resources for other matters.

### BRIEF SUMMARY OF THE INVENTION

It is desirable to provide a system and method for determining the degree of contact between a catheter and a tissue in a body. In particular, it is desirable to be able to determine a degree of contact between a sensor on a distal end of a catheter and a body tissue.

Disclosed herein is a system for displaying contact between a catheter and a tissue in a body. The system generally includes a catheter having a distal portion and a sensor as well as a controller in operable communication with the sensor. The controller is configured to receive as input from the sensor a reading indicative of a degree of contact between the distal portion and the tissue, to calculate a degree of contact between the distal portion of the catheter and the tissue, and to provide an output indicative of the degree of contact.

In some embodiments of the invention, the sensor includes at least one electrode. It is contemplated that the sensor may sense an impedance component, such as an impedance of the tissue in contact with the distal portion of the catheter or an impedance of a catheter component. In other embodiments of the invention, the sensor is a force sensor. Optical sensors are also within the spirit and scope of the invention.

Optionally, the system includes a display coupled to the controller. The controller may then be configured to output a model of the tissue and a model of the catheter to be displayed on the display. The controller may also be configured to display a waveform or meter indicative of the degree of contact on the display.

An appearance of the meter may change as the sensor reading changes. For example, the meter's color may change when the sensor reading crosses a programmable threshold value (e.g., a maximum or minimum acceptable value).

In other embodiments of the invention, the controller is configured to display a beacon projected onto the displayed catheter model. Like the appearance of the meter, the appearance of the beacon (e.g., its color, size, length, intensity, shape, and combinations thereof) may change as the sensor reading changes. Optionally, the controller may also be configured to identify a portion of the sensor in contact with the tissue and to adjust the projected beacon to display an orientation of the catheter relative to the tissue.

In another aspect, the invention provides a method for displaying contact between a catheter and a tissue in a body, including the following steps: providing a catheter having a distal portion and a sensor on the distal portion; providing a controller in communication with a display; establishing communication between the sensor and the controller; acquiring a sensor reading indicative of a degree of contact between the distal portion of the catheter and the tissue; calculating a degree of contact between the sensor and the tissue; and displaying a graphical representation of the calculated degree of contact on the display.

The acquiring step may include measuring an impedance component of the tissue in contact with the distal portion of the catheter. Alternatively, the acquiring step may include measuring an impedance of a catheter component. In turn, the calculating step may include the substep of calculating a composite value for two components of a complex impedance.

Advantageously, a model of the catheter may be displayed on the display, allowing a graphical representation of the calculated degree of contact to be displayed on the model of the catheter.

Also disclosed herein is an article of manufacture, including: a catheter with a sensor; and a computer storage medium having a computer program encoded thereon for determining a degree of contact between a sensor and tissue in a body. The computer program includes computer code for: calculating a degree of contact in response to a signal from the sensor; and generating a visual indicator of the degree of contact to be displayed on a display device.

Optionally, the computer program also includes computer code for determining a location of the catheter in relation to the tissue in the body.

As an additional option, the computer program may include computer code for calculating a location of the tissue and for generating a representation of the tissue to be displayed on the display device. The representation may be, for example, an electro anatomical map of a heart. The computer program may utilize the degree of contact in calculating the location of the tissue. A low pass filter may also be provided to speed the clinician's assessment, for example by smoothing the signal in one or more respects, depending on the application and the needed information.

### BRIEF DESCRIPTION OF THE DRAWINGS

FIG. 1 is diagrammatic view of a system in accordance with the present teachings.

FIG. 2 is a representative display for illustrating impedance measurements in accordance with the present teachings.

FIG. 3A is a representative display for illustrating various degrees of contact using waveform displays in accordance with the present invention.

FIG. 3B is a representative display for illustrating various degrees of contact using a combination of a waveform display and a meter display in accordance with the present invention.

FIG. 3C is a diagrammatic representation of certain signal processing aspects that can be used in accordance with the present invention.

FIGS. 4A-4C are representative displays that illustrate various degrees of contact between a catheter and tissue in accordance with aspects of the present invention.

FIGS. 5A and 5B are additional representative displays that may be used to illustrate various degrees of contact between a catheter and tissue in accordance with aspects of the present invention.

FIGS. 6A-6F are additional representative displays that may be used to illustrate various degrees of contact between a catheter and tissue in accordance with aspects of the present invention.

FIGS. 7A-7D are additional representative displays that may be used to illustrate various degrees of contact between a catheter and tissue in accordance with aspects of the present invention.

FIGS. 8A-8B are representative displays that may be used to illustrate various degrees of contact between multiple electrodes on a catheter and tissue in accordance with aspects of the present invention.

FIG. 9 is a block diagram view of a system in accordance with the present teachings.

FIG. 10 is a yet another graphical representation that can be used to illustrate various degrees of contact between a catheter and a tissue.

FIG. 11 is a simplified schematic view for a system in accordance with the present invention, which may be used for visualization, mapping and navigation of internal body structures.

FIG. 12 is a simplified schematic view illustrating how the present invention may be used to measure complex impedance at the interface of a catheter in contact with tissue.

FIG. 13 is a simplified schematic and block diagram of the three-terminal measurement arrangement of the invention.

### DETAILED DESCRIPTION OF EMBODIMENTS OF THE INVENTION

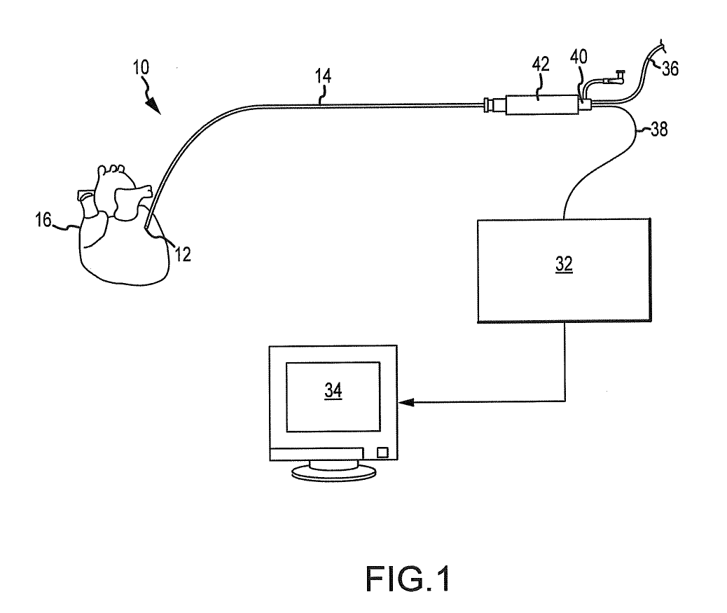

FIG. 1 illustrates one embodiment of a system 10 for providing a more clinically useful display of a degree of contact between a sensor 12 on a catheter 14 and a tissue 16. As used herein, the term “degree of contact” refers to the relationship between sensor 12 and tissue 16; that is, it encompasses not only whether or not sensor 12 and tissue 16 are in contact, but also how hard sensor 12 is pressing into tissue 16. In the illustrated embodiment, tissue 16 comprises heart or cardiac tissue. The present invention may be used to evaluate and display contact between sensors and a variety of body tissues. Further, it should be understood that the present invention may be used to assess a degree of contact between any type of sensor and tissue including, for example, magnetic coils, intracardiac electrodes, ultrasound transducers, optical sensors, force sensors, electrical coupling sensors, needle electrodes, patch electrodes, wet brush electrodes (such as the electrodes disclosed in commonly assigned U.S. application Ser. No. 11/190,724 filed Jul. 27, 2005, the entire disclosure of which is incorporated herein by reference) and virtual electrodes (those formed from a conductive fluid medium such as saline including those disclosed in commonly assigned U.S. Pat. No. 7,326,208, issued Feb. 5, 2008, the entire disclosure of which is incorporated herein by reference).

Catheter 14 includes a handle 42 with a connector 40. In some embodiments, catheter 14 may also include a fluid source 36. Catheter 14 also includes an electrical connection 38 to an electronic control unit 32, which is adapted to provide an output to a display device 34. Electronic control unit 32 includes computer software suitable to receive an input, or reading, from sensor 12, calculate a degree of contact from the input, and to provide an image output to display device 34.

Display device 34 is provided to present the degree of contact in a format useful to the clinician. Display device 34 may also provide a variety of information relating to visualization, mapping and navigation as is known in the art, including, without limitation, measures of electrical signals, two and three dimensional images of the tissue 16 and three-dimensional reconstructions of the tissue 16. Device 34 may comprise an LCD monitor or other conventional display device. In accordance with another aspect of the present invention, the degree of contact may be displayed in one or more ways designed to provide easy interpretation and correlation to tissue contact for the clinician.

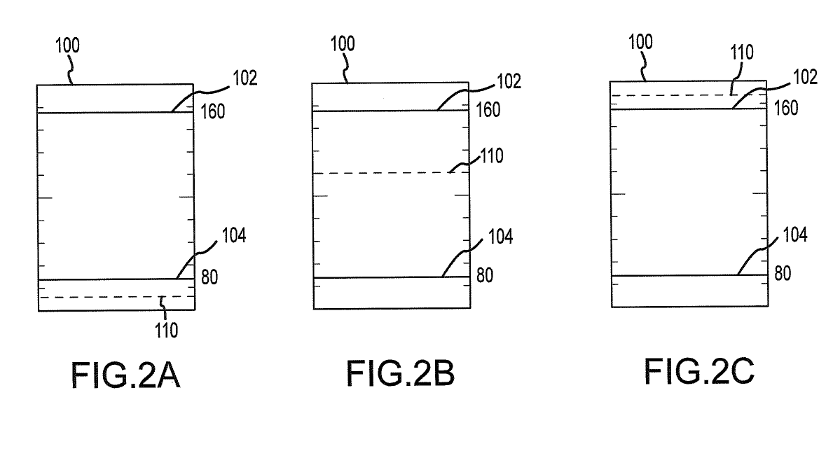

With reference to FIGS. 2A-2C, the image output can be in the form of a meter 100 displayed on display device 34. The meter 100 provides a display of the degree of contact by showing a current value indicator (CVI) 110 of the sensed or computed signal or index. The CVI 110 may be shown alongside an upper boundary 102 and a lower boundary 104.

The upper boundary 102 and lower boundary 104 may be set in advance to values appropriate to the sensor's range. For example, when using a combination of complex impedance values to calculate an electrical coupling index (described in detail below) the meter range may be set to cover 80 ohms to 160 ohms. Likewise, the range could be set by the clinician based on experience or the type of tissue the clinician expects to see during the procedure. In addition, the range can be varied during the procedure, either automatically based on results observed by the system 10, or manually by the clinician.

Meter display 100 can alter the appearance of CVI 110 depending on its relationship to upper boundary 102 and lower boundary 104. For example, CVI 110 can be yellow when below lower boundary 104 (e.g., FIG. 2A), green when between the two boundaries (e.g., FIG. 2B), and red when above upper boundary 102 (e.g., FIG. 2C). Levels above upper boundary 102 may indicate excessive contact, including a danger of perforation. Levels below lower boundary 104 may indicate insufficient contact for mapping purposes or for ablation purposes. As with the color change, the CVI 110 may change shape, size, blink, or otherwise provide a visual indication of its relationship to the boundaries, which may be visible, transparent, or invisible. Likewise the meter display 100 may change in color, shape, size, or appearance depending on the degree of contact. A change in the degree of contact can also be shown by changes in line thickness, changes in fonts, or even in the location of the meter display on display device 34.

In the embodiment shown in FIGS. 2A-2C, two boundaries are shown. In other embodiments any number of additional boundaries can be shown to indicate differing levels of contact. Likewise, only one boundary could be shown.

The meter is advantageously placed close to other physiological displays of interest, such as ablation catheter electrogram traces or a graphical representation of the catheter position on the display device 34.

In a preferred embodiment, the CVI 110 is damped to remove cardiac fluctuation, if present. In particular, fluctuations of the measured signal may interfere with making rapid visual determination of the degree of contact, as the clinician may be forced to watch the meter for an interval and mentally calculate an average. To speed the clinician's assessment, the signal may be smoothed in one or more respects, displaying an average, mean, high, or low, depending on the application and the needed information. For example, a low pass filter may be employed to dampen the meter display. A FIR (Finite Impulse Response) low pass filter running the average over 1 second may be employed. It is anticipated that the upper and lower boundaries 102, 104 would need to be adjusted to the specific filters used.

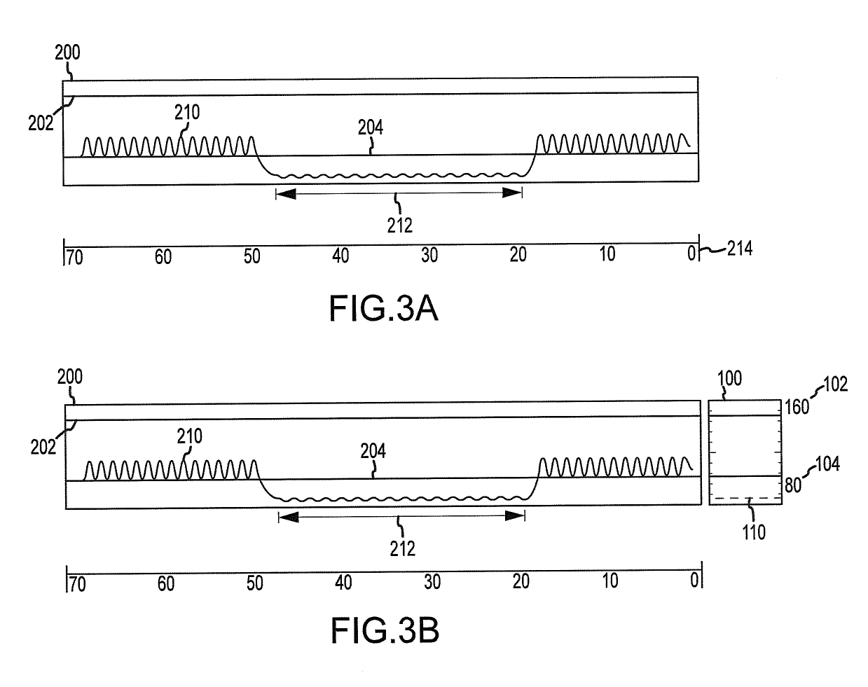

With reference to FIGS. 3A-3B, the image output can also be in the form of a waveform display 200 displayed on display device 34. The waveform display 200 provides a display of the degree of contact by showing a waveform 210 of the sensed or computed signal or index. The waveform 210 may be shown alongside an upper boundary 202 and a lower boundary 204.

As shown in FIG. 3A, contact may be shown as a reduced waveform 212, a smoothed waveform, or an increased waveform (not shown). The degree of contact can be demonstrated over a time frame, 5-10 seconds or 150-300 seconds. The shown time frame 214 can be preset, varied depending on the procedure, or adjusted by the clinician during the procedure as needed to show the particular physiologic events of interest. In addition, the waveform can be frozen for study, fixed at a screen position until a rolling update refreshes the image with the latest signal (as is commonly done with ECG signals) or can be left in a rolling update format that refreshes the image with the latest signal. As with meter 100, the waveform display 200, waveform 210, and boundaries 202, 204 can be varied in color, shape, location, appearance, and the like as described above.

FIG. 3B shows the waveform display 200 alongside a meter 100 to provide both a time frame for historical reference and a current meter visualization. Other combinations are likewise possible.

As can be appreciated from the fluctuations shown in FIG. 3A, the fluctuation amplitude contains valuable information. In a conventional EP monitoring system, the GE Prucka CardioLab® EP System, zooming out to see 120 or 200 seconds results in the display of subsampled signals. This can lead to an incorrect perception that the fluctuation amplitude is lower in some spots and of excessive variation of amplitude over time as subsamples fall at various points on the waveform. This subsampling results from software that deals with very long intervals taken at a high sample rate by simply skipping data points and not sending each of the points (120 seconds of 1200 Hz data, or 144,000 points) to a display plot routine.

In contrast, with reference to FIG. 3C, a preferred embodiment of the waveform starts with signal 210 uses a filtering and decimation stage 220 to reduce the data sampling rate 215 and signal bandwidth to obtain an accurate representation of signal amplitude. In particular, this can be accomplished by band limiting the signals to 20 Hz and decimated to 100 Hz (225). This would reduce the total number of points to 12,000 for a similar 120 second signal. In addition, a circular buffer 230 can be applied to further modify the signal, before electronic control unit 32 outputs 235 an image 240 to display device 34. In some situations where band limiting would itself alter signal amplitude, a subsample can be taken that would retain the maximum and minimum values over the slower and longer intervals. The system 10 can subsample to 50 Hz but keep two numeric arrays with both extremes for each signal.

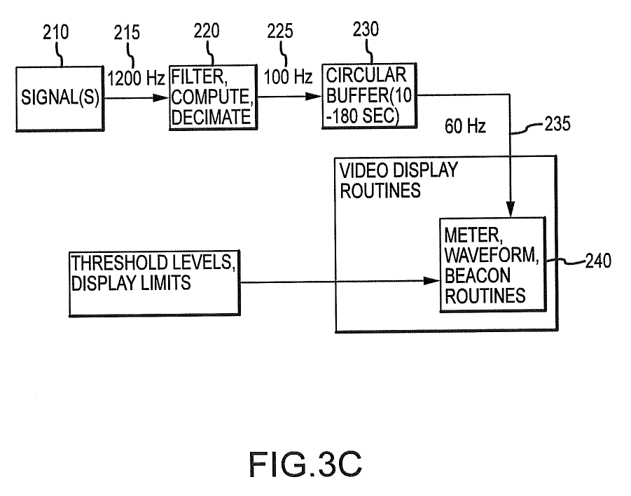

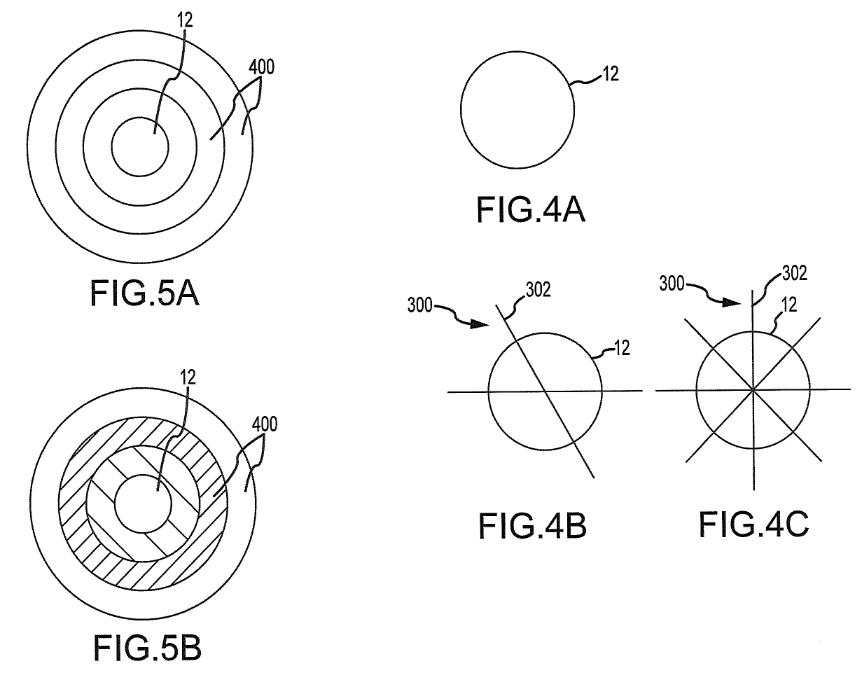

A third display type can take advantage of a catheter image or representation on any currently existing display, fluoroscopy, ICE, electro anatomical mapping, CT, MRI, or the like. The catheter image and particularly sensor 12 are depicted without contact, or with a degree of contact below a threshold value, in FIG. 4A. FIG. 4B shows lines 302 emanating from sensor 12 to indicate a first threshold contact value has been exceeded, and FIG. 4C shows additional lines 302 to show that a second threshold value has been exceeded. Similarly, FIG. 5A shows the sensor 12 with concentric rings 400 around the catheter. As the degree of contact is increased, the rings 400 may become shaded, as shown in FIG. 5B. The shading may increase or decrease as the degree of contact changes. The color may likewise change from green to show low contact to red to show increased, excessive contact. Additional rings may be added or subtracted as the degree of contact increases or decreases.

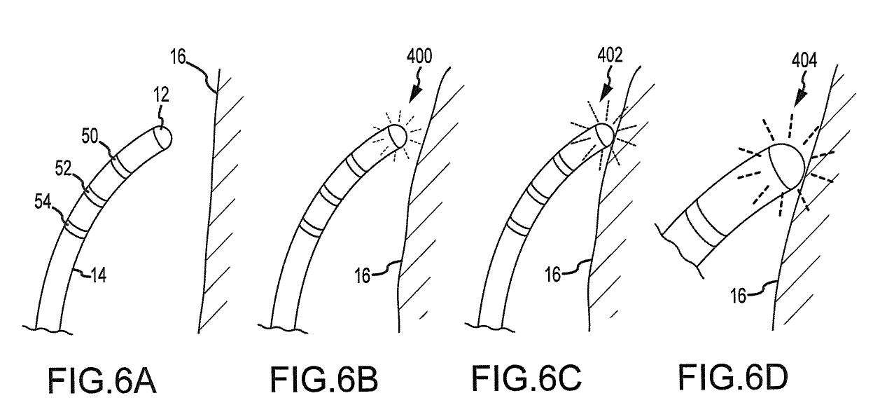

FIG. 6A shows a catheter 14, with sensor 12 and electrodes 50, 52, and 54 in a state of no contact. As shown in FIGS. 6B-D, as the sensor 12 begins to approach a tissue, contact, proximity, or electrical coupling can be demonstrated by lines 400 emanating from the sensor 12 or the catheter 14. As contact grows stronger, the longer lines 402 can be displayed, or bolder lines 404 can be displayed. Likewise, lines 400 can change color, shape, can move or blink, or otherwise alter their appearance to demonstrate the degree of contact. As shown in FIG. 6E, lines 406 may indicate the orientation of the contact by only appearing on the portion of the catheter 14 or sensor 12 that is in contact with the tissue 16. In FIGS. 6A-E lines 400-406 are shown as increasing in length as the degree of contact increases, but likewise the lines can decrease in length to show decreasing distance to the tissue 16. For example, as shown in FIG. 6F, lines 408 can be longest where there is contact, shorter where there is near contact, and absent where the probe is far from contact. The length of the lines can approximate the distance between the sensor 12 and the tissue 16, or can merely be scaled to the degree of contact read by the sensor.

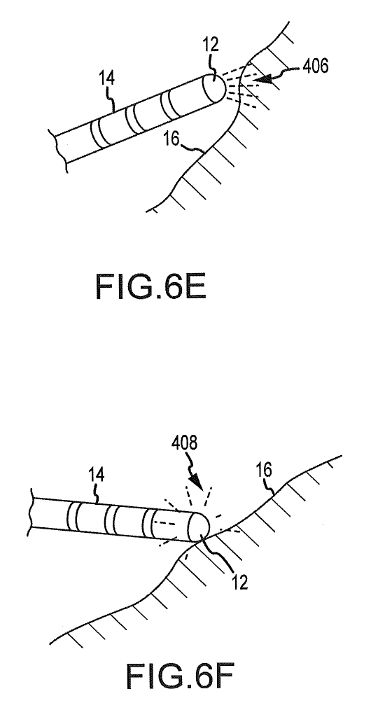

FIGS. 7A-D show an additional “gas tank gauge” embodiment. In this embodiment a gauge 500 reads “empty” when the degree of contact is absent or low. As contact increases, as shown in FIG. 7B, the gauge 500 begins to fill. As shown in FIG. 7C, at strong or excessive contact, the gauge 500 may be full. Likewise, the degree of contact may be shown by the gauge 500 changing appearance, color, or size. As shown in FIG. 7D, multiple colors or shadings can be used as well. In any of the above embodiments the catheter and sensor can be shown alone or in combination, and in either 2D or in 3D.

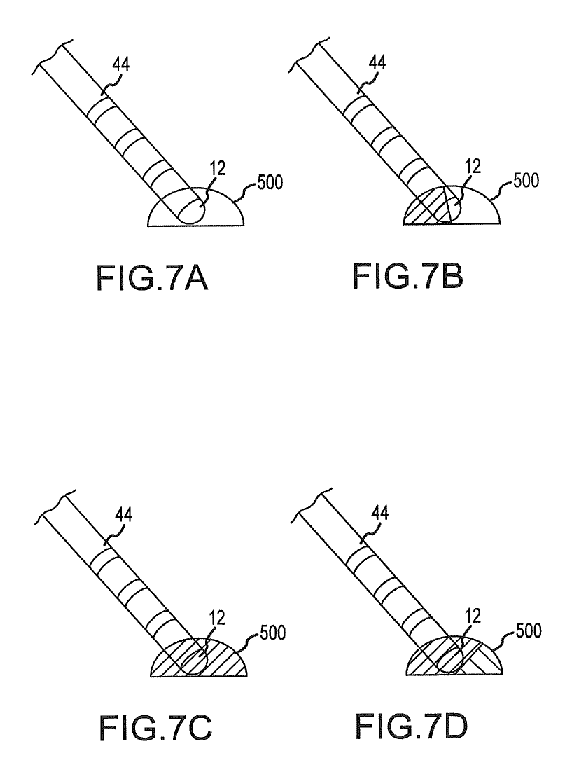

The present invention may also be used to illustrate a degree of contact for multiple electrodes. As shown in FIG. 8A, catheter 14 may have multiple electrodes, including tip electrode 12 and band electrodes 50, 52, and 54. If, as depicted in FIG. 8A, the catheter tip electrode is in strong contact, while the other electrodes are at varying distances from tissue 16, strong contact can be shown at the tip electrode by lines 402, while—if relevant—weaker contact can be shown at another electrode (electrode 50) by lines 400. Likewise, when multiple electrodes 12, 50, 52, and 54 are all in varying degrees of contact with tissue 16, lines 402 can illustrate the relevant degree of contact for each electrode.

A preferred embodiment of system 10 is used within an electro anatomical mapping system. In a preferred embodiment, the electro anatomical mapping system is the EnSite NavX™ navigation and visualization system of St. Jude Medical, Atrial Fibrillation Division, Inc., which generates electrical fields. Other localization systems, however, may be used in connection with the present invention, including for example, the CARTO navigation and location system of Biosense Webster, Inc., the AURORA® system of Northern Digital Inc., or Stereotaxis' NIOBE® Magnetic Navigation System, all of which utilize magnetic fields rather than electrical fields. The localization and mapping systems described in the following patents (all of which are hereby incorporated by reference in their entireties) can also be used with the present invention: U.S. Pat. Nos. 6,990,370; 6,978,168; 6,947,785; 6,939,309; 6,728,562; 6,640,119; 5,983,126; and 5,697,377.

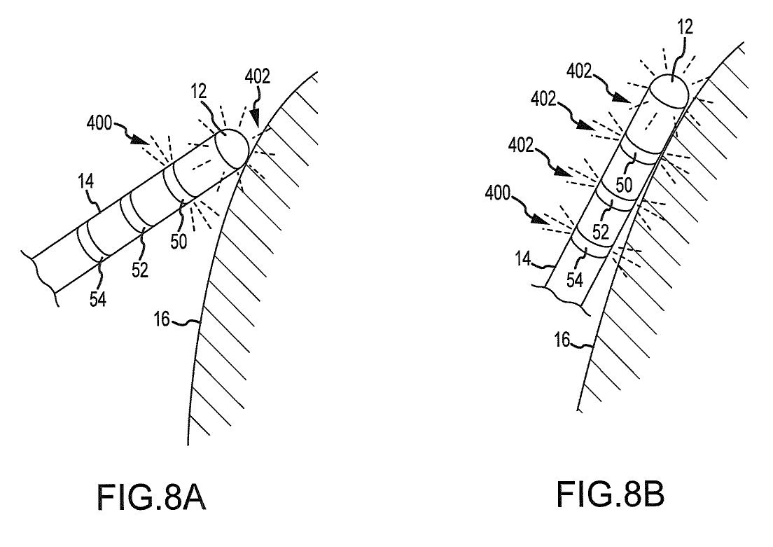

FIG. 9 shows a schematic diagram of a localization system 8 for conducting cardiac electrophysiology studies by navigating a cardiac catheter and measuring electrical activity occurring in a heart 16 of a patient 11 and three-dimensionally mapping the electrical activity and/or information related to or representative of the electrical activity so measured. System 8 can be used, for example, to create an anatomical model of the patient's heart 16 using one or more electrodes or other sensors, such as magnetic coils. System 8 can also be used to measure electrophysiology data at a plurality of points along a cardiac surface, and store the measured data in association with location information for each measurement point at which the electrophysiology data was measured, for example to create a diagnostic data map of the patient's heart 10. As one of ordinary skill in the art will recognize, and as will be further described below, localization system 8 determines the location of objects, typically within a three-dimensional space, and expresses those locations as position information determined relative to at least one reference.

For simplicity of illustration, the patient 11 is depicted schematically as an oval. In the embodiment shown in FIG. 9, three sets of surface electrodes (patch electrodes) are shown applied to a surface of the patient 11, defining three generally orthogonal axes, referred to herein as an x-axis, a y-axis, and a z-axis. In other embodiments the electrodes could be positioned in other arrangements, for example multiple electrodes on a particular body surface. Likewise, the electrodes do not need to be on the body surface, but could be fixed on an external apparatus, or electrodes positioned internally to the body could be used.

In FIG. 9, the x-axis surface electrodes 4, 6 are applied to the patient along a first axis, such as on the lateral sides of the thorax region of the patient (applied to the patient's skin underneath each arm) and may be referred to as the Left and Right electrodes. The y-axis electrodes 18, 19 are applied to the patient along a second axis generally orthogonal to the x-axis, such as along the inner thigh and neck regions of the patient, and may be referred to as the Left Leg and Neck electrodes. The z-axis electrodes 22, 23 are applied along a third axis generally orthogonal to both the x-axis and the y-axis, such as along the sternum and spine of the patient in the thorax region, and may be referred to as the Chest and Back electrodes. The heart 16 lies between these pairs of surface electrodes 4/6, 18/19, and 23/22.

An additional surface reference electrode (a “belly patch”) 21 provides a reference and/or ground electrode for the system 8. The belly patch electrode 21 may be an alternative to a fixed intra-cardiac electrode 31, described in further detail below. It should also be appreciated that, in addition, the patient 11 may have most or all of the conventional electrocardiogram (“ECG”) system leads in place. This ECG information is available to the system 8, although not illustrated in FIG. 9.

A representative catheter 13 having at least one sensor 17 (a distal electrode) is also shown. This representative catheter electrode 17 is referred to as the “roving electrode,” “moving electrode,” or “measurement electrode” throughout the specification. Typically, multiple electrodes on catheter 13, or on multiple such catheters, will be used. In one embodiment, for example, localization system 8 may comprise sixty-four electrodes on twelve catheters disposed within the heart and/or vasculature of the patient. Of course, this embodiment is merely exemplary, and any number of electrodes and catheters may be used within the scope of the present invention.

An optional fixed reference electrode 31 (attached to a wall of the heart 16) is shown on a second catheter 29. For calibration purposes, this electrode 31 may be stationary (attached to or near the wall of the heart) or disposed in a fixed spatial relationship with the roving electrodes (electrode 17), and thus may be referred to as a “navigational reference” or “local reference.” The fixed reference electrode 31 may be used in addition or alternatively to the surface reference electrode 21 described above. In many instances, a coronary sinus electrode or other fixed electrode in the heart 16 can be used as a reference for measuring voltages and displacements; that is, as described below, fixed reference electrode 31 may define the origin of a coordinate system.

Each surface electrode is coupled to the multiplex switch 24, and the pairs of surface electrodes are selected by software running on a computer 32, which couples the surface electrodes to a signal generator 25. The computer 32, for example, may comprise a conventional general-purpose computer, a special-purpose computer, a distributed computer, or any other type of computer. The computer 32 may comprise one or more processors, such as a single central processing unit (CPU), or a plurality of processing units, commonly referred to as a parallel processing environment, which may execute instructions to practice the various aspects of the present invention described herein.

Generally, three nominally orthogonal electric fields are generated by a series of driven and sensed electric dipoles (surface electrode pairs 4/6, 18/19, and 22/23) in order to realize catheter navigation in a biological conductor. Alternatively, these orthogonal fields can be decomposed and any pairs of surface electrodes can be driven as dipoles to provide effective electrode triangulation. Likewise, the electrodes 4, 6, 18, 19, 22, and 23 (or any number of electrodes) could be positioned in any other effective arrangement for driving a current to or sensing a current from an electrode in the heart. For example, multiple electrodes could be placed on the back, sides, and/or belly of patient 11. Additionally, such non-orthogonal methodologies add to the flexibility of the system. For any desired axis, the potentials measured across the roving electrodes resulting from a predetermined set of drive (source-sink) configurations may be combined algebraically to yield the same effective potential as would be obtained by simply driving a uniform current along the orthogonal axes.

Thus, any two of the surface electrodes 4, 6, 18, 19, 22, 23 may be selected as a dipole source and drain with respect to a ground reference, such as belly patch 21, while the unexcited electrodes measure voltage with respect to the ground reference. The roving electrode 17 placed in the heart 10 is exposed to the field from a current pulse and are measured with respect to ground, such as belly patch 21. In practice the catheters within the heart may contain more electrodes than shown, and each electrode potential may be measured. As previously noted, at least one electrode may be fixed to the interior surface of the heart to form a fixed reference electrode 31, which is also measured with respect to ground, such as belly patch 21, and which may be defined as the origin of the coordinate system relative to which localization system 8 measures positions. Data sets from each of the surface electrodes, the internal electrodes, and the virtual electrodes may all be used to determine the location of the roving electrode 17 within heart 16.

The measured voltages may be used to determine the location in three-dimensional space of the electrodes inside the heart, such as roving electrode 17 relative to a reference location, such as reference electrode 31. That is, the voltages measured at reference electrode 31 may be used to define the origin of a coordinate system, while the voltages measured at roving electrode 17 may be used to express the location of roving electrode 17 relative to the origin. Preferably, the coordinate system is a three-dimensional (x, y, z) Cartesian coordinate system, though the use of other coordinate systems, such as polar, spherical, and cylindrical coordinate systems, is within the scope of the invention.

As should be clear from the foregoing discussion, the data used to determine the location of the electrode(s) within the heart is measured while the surface electrode pairs impress an electric field on the heart. The electrode data may also be used to create a respiration compensation value used to improve the raw location data for the electrode locations as described in U.S. Patent Application Publication No. 2004/0254437, which is hereby incorporated herein by reference in its entirety. The electrode data may also be used to compensate for changes in the impedance of the body of the patient as described in co-pending U.S. patent application Ser. No. 11/227,580, filed on 15 Sep. 2005, which is also incorporated herein by reference in its entirety.

In summary, the system 8 first selects a set of surface electrodes and then drives them with current pulses. While the current pulses are being delivered, electrical activity, such as the voltages measured at least one of the remaining surface electrodes and in vivo electrodes, is measured and stored. Compensation for artifacts, such as respiration and/or impedance shifting, may be performed as indicated above.

The fields generated by localization system 8, whether an electrical field (e.g., EnSite NavX™), a magnetic field (e.g., CARTO, AURORA®, NIOBE®), or another suitable field may be referred to generically as “localization fields,” while the elements generating the fields, such as surface electrodes 4, 6, 18, 19, 22, and 23 may be generically referred to as “localization field generators.” As described above, surface electrodes 4, 6, 18, 19, 22, and 23 may also function as detectors to measure the characteristics of the localization field (the voltages measured at roving electrodes 17, 50, 52, 54, or a current from roving electrodes 17, 50, 52, 54), and thus may also be referred to as “localization elements.” Though the present invention will be described primarily in the context of a localization system that generates an electrical field, one of ordinary skill in the art will understand how to apply the principles disclosed herein in other types of localization fields, and in particular other types of non-ionizing localization fields (by replacing electrodes with coils to detect different components of a magnetic field).

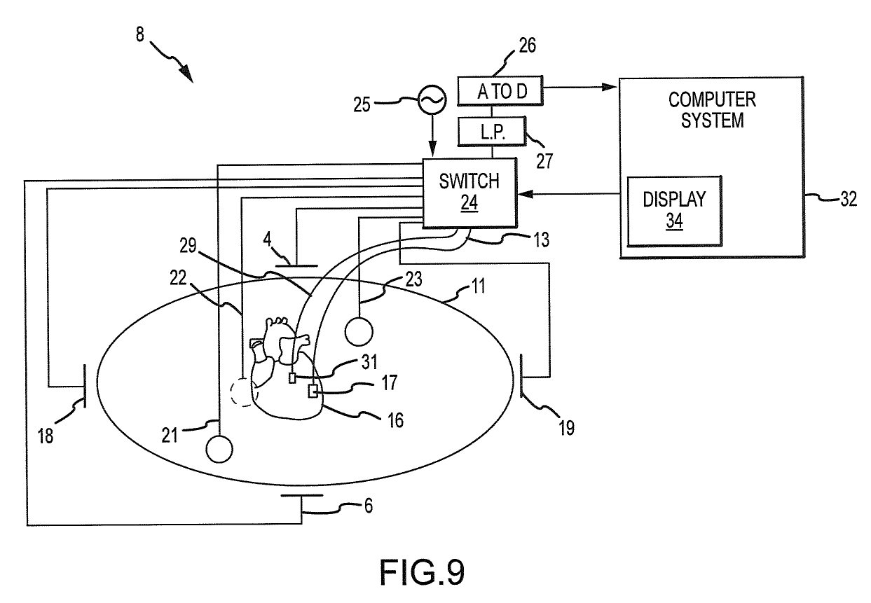

When system 10 is used with a system 8 that creates localization field, the degree of coupling can be illustrated on the screen in relation to the anatomical map generated. FIG. 10 shows an electroanatomical map with two catheter representations 14 imposed on it. As shown, the linear catheter 14 visually indicates a degree of contact via lines 402. Likewise, waveform 200 and meter 100 are shown in close proximity to the model of the heart 116. This allows the clinician ready visualization of the degree of contact at the same moment he views its location, reducing his workload.

Visualization of the degree of contact can facilitate catheter manipulation techniques that more efficiently build a geometry. When the clinician understands where he has been in contact and where he has not been in contact, he is better able to manipulate the catheter to additional “exterior” points, those points that form the exterior boundaries of the tissue, the heart chamber being investigated. As initial maps are being generated, a screen image can be provided to the clinician that visually indicates which points were taken at a location that was in contact with the tissue, by coloring such points green while interior points are colored red. Likewise, a surface generated can be colored a particular color depending on the degree of contact from the points used in the surface.

This invention can also result in improved geometry creation by virtue of reducing the number of interior points collected, or by helping system 8 label such points as interior points due to the lack of contact. The 3-D surface rendering algorithms can take this data into account in building a shell around the outermost points.

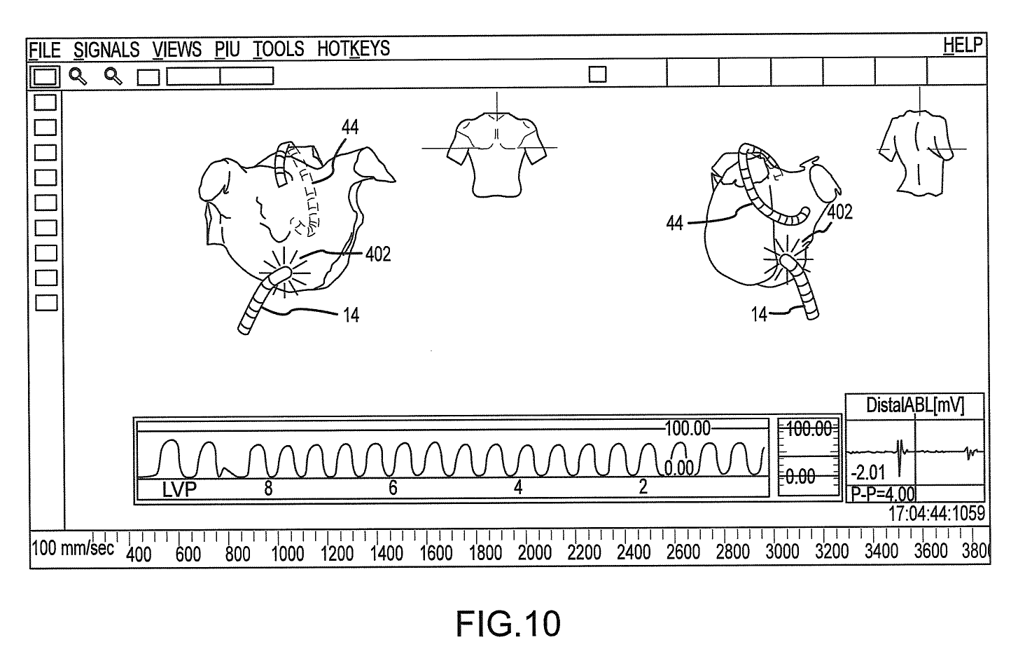

With reference to FIG. 11, system 10 may include patch electrodes 4, 6, 18, an ablation generator 124, a tissue sensing circuit 126, an electrophysiology (EP) monitor 34 and a system 8 for visualization, mapping and navigation of internal body structures which may include an electronic control unit 32 in accordance with the present invention and a display device 34 among other components.

Catheter 14 is provided for examination, diagnosis and treatment of internal body tissues such as tissue 16. In accordance with one embodiment of the invention, catheter 14 comprises an ablation catheter and, more particularly, an irrigated radio-frequency (RF) ablation catheter. It should be understood, however, that the present invention can be implemented and practiced regardless of the type of ablation energy provided (cryoablation, ultrasound, etc.) Catheter 14 is connected to a fluid source 36 having a biocompatible fluid such as saline through a pump 37 (which may comprise, for example, a fixed rate roller pump or variable volume syringe pump with a gravity feed supply from fluid source 36 as shown) for irrigation. Catheter 14 is also electrically connected to ablation generator 124 for delivery of RF energy. Catheter 14 may include a cable connector or interface 40, a handle 42, a shaft 44 and one or more electrodes 12, 50, 52. Catheter 14 may also include other conventional components not illustrated herein such as a temperature sensor, additional electrodes, and corresponding conductors or leads.

Connector 40 provides mechanical, fluid and electrical connection(s) for cable 38 and fluid source 36. Connector 40 is conventional in the art and is disposed at a proximal end of catheter 14.

Handle 42 provides a location for the clinician to hold catheter 14 and may further provides means for steering or guiding shaft 44 within patient 11. For example, handle 42 may include means to change the length of a guidewire extending through catheter 14 to distal end 48 of shaft 44 to steer shaft 44. Handle 42 is also conventional in the art and it will be understood that the construction of handle 42 may vary.

Shaft 44 is an elongated, tubular, flexible member configured for movement within the body. Shaft 44 supports electrodes 12, 50, 52 associated conductors, and possibly additional electronics used for signal processing or conditioning. Shaft 44 may also permit transport, delivery and/or removal of fluids (including irrigation fluids and bodily fluids), medicines, and/or surgical tools or instruments. Shaft 44 may be made from conventional materials such as polyurethane and defines one or more lumens configured to house and/or transport electrical conductors, fluids or surgical tools. Shaft 44 may be introduced into a blood vessel or other structure within the body through a conventional introducer. Shaft 44 may then be steered or guided through the body to a desired location such as tissue 16 with guide wires or other means known in the art.

Electrodes 12, 50, 52 are provided for a variety of diagnostic and therapeutic purposes including, for example, electrophysiological studies, catheter identification and location, pacing, cardiac mapping and ablation. In the illustrated embodiment, catheter includes an ablation tip electrode 12 and a pair of ring electrodes 50, 52. It should be understood, however, that the number, orientation and purpose of electrodes 12, 50, 52 may vary.

Patch electrodes 4, 6, 18 provide RF or navigational signal injection paths and/or are used to sense electrical potentials. Electrodes 4, 6, 18 may also have additional purposes such as the generation of an electromechanical map. Electrodes 4, 6, 18 are made from flexible, electrically conductive material and are configured for affixation to patient 11 such that electrodes 4, 6, 18 are in electrical contact with the patient's skin. Electrode 18 may function as an RF indifferent/dispersive return for the RF ablation signal.

Ablation generator 124 generates, delivers and controls RF energy used by ablation catheter 14. Generator 124 is conventional in the art and may comprise the commercially available unit sold under the model number IBI-1500T RF Cardiac Ablation Generator, available from Irvine Biomedical, Inc. Generator 124 includes an RF ablation signal source 154 configured to generate an ablation signal that is output across a pair of source connectors: a positive polarity connector SOURCE (+) which may connect to tip electrode 12; and a negative polarity connector SOURCE(−) which may be electrically connected by conductors or lead wires to one of patch electrodes 4, 6, 18. It should be understood that the term connectors as used herein does not imply a particular type of physical interface mechanism, but is rather broadly contemplated to represent one or more electrical nodes. Source 154 is configured to generate a signal at a predetermined frequency in accordance with one or more user specified parameters (power, time, etc.) and under the control of various feedback sensing and control circuitry as is know in the art. Source 154 may generate a signal, for example, with a frequency of about 450 kHz or greater. Generator 124 may also monitor various parameters associated with the ablation procedure including impedance, the temperature at the tip of the catheter, ablation energy and the position of the catheter and provide feedback to the clinician regarding these parameters. The impedance measurement output by generator 124, however, reflects the magnitude of impedance not only at tissue 16, but the entire impedance between tip electrode 12 and the corresponding patch electrode 18 on the body surface. The impedance output by generator 124 is also not easy to interpret and correlate to tissue contact by the clinician.

Tissue sensing circuit 126 provides a means, such as tissue sensing signal source 156, for generating an excitation signal used in impedance measurements and means, such as complex impedance sensor 158, for resolving the detected impedance into its component parts. Signal source 156 is configured to generate an excitation signal across source connectors SOURCE (+) and SOURCE (−). Source 156 may output a signal having a frequency within a range from about 1 kHz to over 500 kHz, more preferably within a range of about 2 kHz to 200 kHz, and even more preferably about 20 kHz. In one embodiment, the excitation signal is a constant current signal, preferably in the range of between 20-200 μA, and more preferably about 100 μA. As discussed below, the constant current AC excitation signal generated by source 156 is configured to develop a corresponding AC response voltage signal that is dependent on the complex impedance of tissue 16 and is sensed by complex impedance sensor 158. Sensor 158 resolves the complex impedance into its component parts (the resistance (R) and reactance (X) or the impedance magnitude (|Z|) and phase angle (<Z or φ)). Sensor 158 may include conventional filters (bandpass filters) to block frequencies that are not of interest, but permit appropriate frequencies, such as the excitation frequency, to pass as well as conventional signal processing software used to obtain the component parts of the measured complex impedance.

It should be understood that variations are contemplated by the present invention. For example, the excitation signal may be an AC voltage signal where the response signal comprises an AC current signal. Nonetheless, a constant current excitation signal is preferred as being more practical. It should be appreciated that the excitation signal frequency is preferably outside of the frequency range of the RF ablation signal, which allows the complex impedance sensor 158 to more readily distinguish the two signals, and facilitates filtering and subsequent processing of the AC response voltage signal. The excitation signal frequency is also preferably outside the frequency range of conventionally expected electrogram (EGM) signals in the frequency range of 0.05-1 kHz. Thus, in summary, the excitation signal preferably has a frequency that is preferably above the typical EGM signal frequencies and below the typical RF ablation signal frequencies.

Circuit 126 is also connected, for a purpose described hereinbelow, across a pair of sense connectors: a positive polarity connector SENSE (+) which may connect to tip electrode 12; and a negative polarity connector SENSE (−) which may be electrically connected to one of patch electrodes 4, 6, 18. It should again be understood that the term connectors as used herein does not imply a particular type of physical interface mechanism, but is rather broadly contemplated to represent one or more electrical nodes.

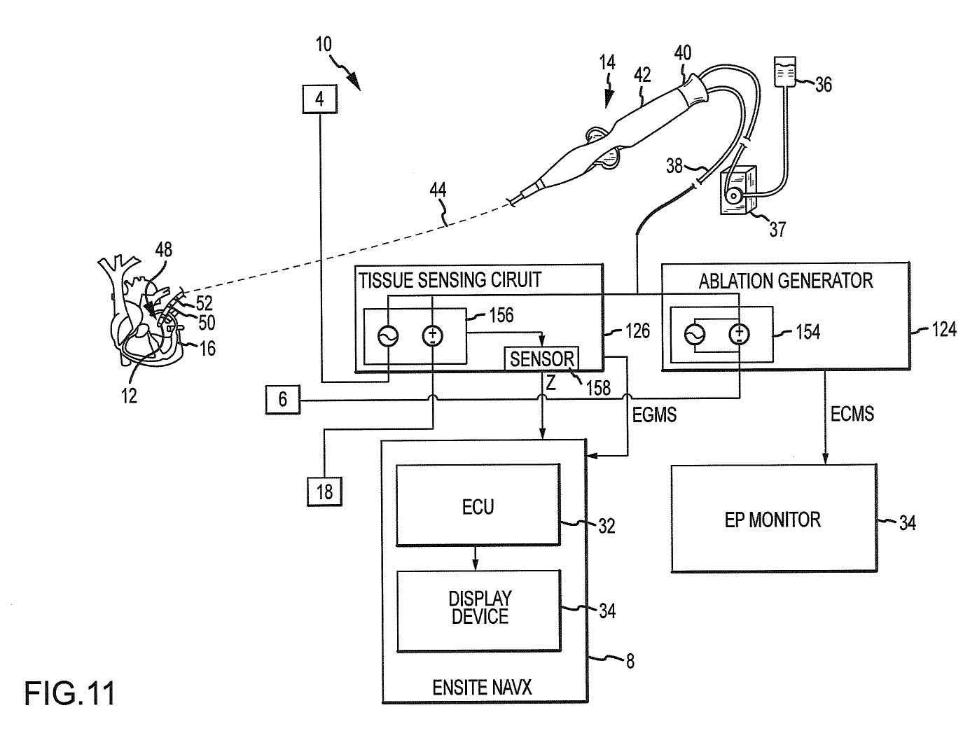

Referring now to FIG. 12, connectors SOURCE (+), SOURCE (−), SENSE (+) and SENSE (−) from a three terminal arrangement permitting measurement of the complex impedance at the interface of tip electrode 12 and tissue 16. Complex impedance can be expressed in rectangular coordinates as set forth in equation (1):

Z=R+jX (1)

where R is the resistance component (expressed in ohms); and X is a reactance component (also expressed in ohms). Complex impedance can also be expressed polar coordinates as set forth in equation (2):

Z=r·e^{jθ}=|Z|·e^{j<Z} (2)

where |Z| is the magnitude of the complex impedance (expressed in ohms) and <Z=θ is the phase angle expressed in radians. Alternatively, the phase angle may be expressed in terms of degrees where

$$φ = ( \frac{180}{π} ) θ .$$

Throughout the remainder of this specification, phase angle will be preferably referenced in terms of degrees. The three terminals comprise: (1) a first terminal designated “A-Catheter Tip” which is the tip electrode 12; (2) a second terminal designated “B-Patch 1” such as source return patch electrode 4; and (3) a third terminal designated “C-Patch 2” such as the sense return patch electrode 18. In addition to the ablation (power) signal generated by source 154 of ablation generator 124, the excitation signal generated by source 156 in tissue sensing circuit 126 is also be applied across the source connectors (SOURCE (+), SOURCE (−)) for the purpose of inducing a response signal with respect to the load that can be measured and which depends on the complex impedance. As described above, in one embodiment, a 20 kHz, 100 μA AC constant current signal is sourced along the path 160, as illustrated, from one connector (SOURCE (+), starting at node A) through the common node (node D) to a return patch electrode (SOURCE(−), node B). The complex impedance sensor 158 is coupled to the sense connectors (SENSE (+), SENSE (−)), and is configured to determine the impedance across the path 162. For the constant current excitation signal of a linear circuit, the impedance will be proportional to the observed voltage developed across SENSE (+)/SENSE(−), in accordance with Ohm's Law: Z=V/I. Because voltage sensing is nearly ideal, the current flows through the path 160 only, so the current through path 162 (node D to node C) due to the excitation signal is effectively zero. Accordingly, when measuring the voltage along path 162, the only voltage observed will be where the two paths intersect (i.e. from node A to node D). Depending on the degree of separation of the two patch electrodes (i.e., those forming nodes B and C), an ever-increasing focus will be placed on the tissue volume nearest the tip electrode 12. If the patch electrodes are physically close to each other, the circuit pathways between the catheter tip electrode 12 and the patch electrodes will overlap significantly and impedance measured at the common node (i.e., node D) will reflect impedances not only at the interface of the catheter electrode 12 and tissue 16, but also other impedances between tissue 16 and the surface of body. As the patch electrodes are moved further part, the amount of overlap in the circuit paths decreases and impedance measured at the common node is only at or near the tip electrode 12 of catheter 14.

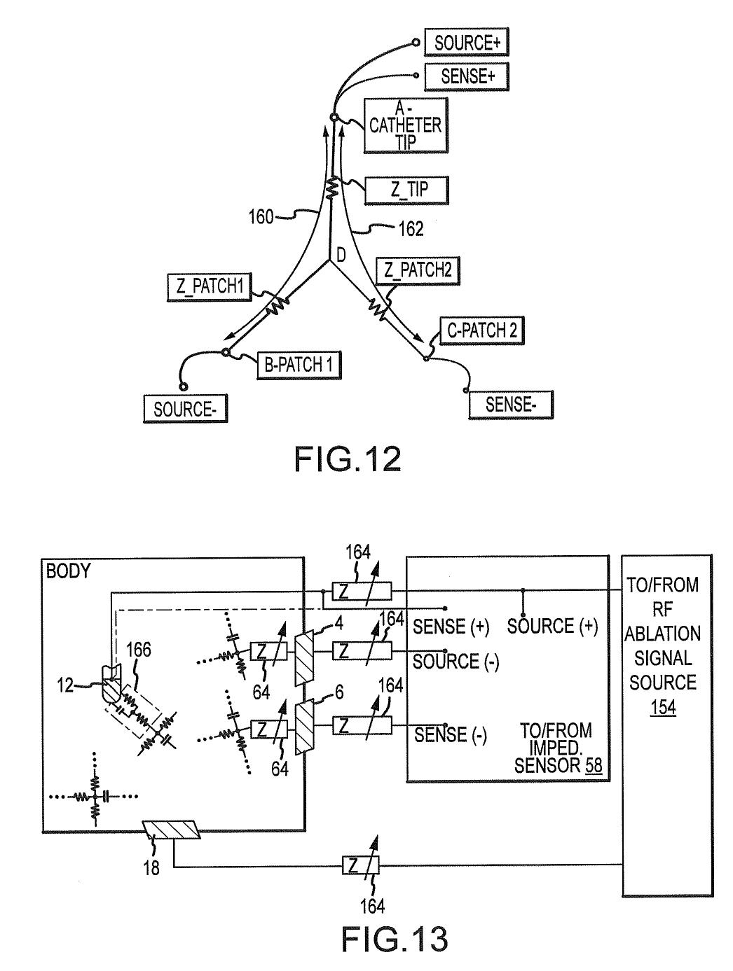

Referring now to FIG. 13, the concept illustrated in FIG. 12 is extended. FIG. 13 is a simplified schematic and block diagram of the three-terminal measurement arrangement of the invention. For clarity, it should be pointed out that the SOURCE (+) and SENSE (+) lines may be joined in the catheter connector 40 or handle 42 (as in solid line) or may remain separate all the way to the tip electrode (the SENSE (+) line being shown in phantom line from the handle 42 to the tip electrode 12). FIG. 13 shows in particular several sources of complex impedance variations, shown generally as blocks 164, that are considered “noise” because such variations do not reflect the physiologic changes in the tissue 16 or electrical coupling whose complex impedance is being measured. For reference, the tissue 16 whose complex impedance is being measured is that near and around the tip electrode 12 and is enclosed generally by a phantom-line box 166 (and the tissue 16 is shown schematically, in simplified form, as a resistor/capacitor combination). One object of the invention is to provide a measurement arrangement that is robust or immune to variations that are not due to changes in or around box 166. For example, the variable complex impedance boxes 64 that are shown in series with the various cable connections (e.g., in the SOURCE (+) connection, in the SOURCE (−) and SENSE (−) connections, etc.) may involve resistive/inductive variations due to cable length changes, cable coiling and the like. The variable complex impedance boxes 164 that are near the patch electrodes 4, 6, may be more resistive/capacitive in nature, and may be due to body perspiration and the like over the course of a study. As will be seen, the various arrangements of the invention are relatively immune to the variations in blocks 64, exhibiting a high signal-to-noise (S/N) ratio as to the complex impedance measurement for block 66.

Although the SOURCE (−) and SENSE (−) returns are illustrated in FIG. 13 as patch electrodes 4, 6, it should be understood that other configurations are possible. In particular, indifferent/dispersive return electrode 18 can be used as a return as well as another electrode 50, 52 on catheter 14, such as ring electrode 50 as described in commonly assigned U.S. application Ser. No. 11/966,232, filed Dec. 28, 2007, the entire disclosure of which is incorporated herein by reference.

EP monitor 34 is provided to display electrophysiology data including, for example, an electrogram. Monitor 34 is conventional in the art and may comprise an LCD or CRT monitor or another conventional monitor. Monitor 34 may receive inputs from ablation generator 124 as well as other conventional EP lab components not shown in the illustrated embodiment.

Electronic Control Unit (ECU) 32 is provided to acquire values for first and second components of a complex impedance between the catheter tip electrode 12 and tissue 16 and to calculate a coupling index responsive to the values with the coupling index indicative of a degree of coupling between electrode 12 and tissue 16. ECU 32 preferably comprises a programmable microprocessor or microcontroller, but may alternatively comprise an application specific integrated circuit (ASIC). ECU 32 may include a central processing unit (CPU) and an input/output (I/O) interface through which ECU 32 may receive a plurality of input signals including signals from sensor 158 of tissue sensing circuit 126 and generate a plurality of output signals including those used to control display device 34. In accordance with one aspect of the present invention, ECU 32 may be programmed with a computer program (i.e., software) encoded on a computer storage medium for determining a degree of coupling between an electrode on a catheter and tissue in a body. The program includes code for calculating a coupling index responsive to values for first and second components of the complex impedance between the catheter electrode 12 and tissue 16 with the coupling index indicative of a degree of coupling between the catheter electrode 12 and the tissue 16.

ECU 32 acquires one or more values for two component parts of the complex impedance from signals generated by sensor 158 of tissue sensing circuit 126 (i.e., the resistance (R) and reactance (X) or the impedance magnitude (|Z|) and phase angle (θ) or any combination of the foregoing or derivatives or functional equivalents thereof). In accordance with one aspect of the present invention, ECU 32 combines values for the two components into a single coupling index that provides an improved measure of the degree of coupling between electrode 12 and tissue 16 and, in particular, the degree of electrical coupling between electrode 12 and tissue 16.

The present invention may also be used as a proximity sensor. As an electrode such as electrode 12 approaches tissue 16 the impedance changes as does the degree of contact. Further, for some electrode configurations, this change is independent of the angle at which the electrode is 12 is disposed relative to tissue 16. The degree of contact is therefore indicative of the proximity of the electrode 12 to tissue 16. In some applications, the general position (with a frame of reference) and speed of the tip of catheter 14 and electrode 12 is known (although the proximity of electrode 12 to tissue 16 is unknown). This information can be combined to define a value (the “degree of contact rate of change”) that is indicative of the rate of change in the degree of contact as electrode 12 approaches tissue 16 and which may provide an improved measure of the proximity of the electrode 12 to tissue 16. This information can be used, for example, in robotic catheter applications to slow the rate of approach prior to contact and also in connection with a transseptal access sheath having a distal electrode to provide an indication that the sheath is approaching (and/or slipping away from) the septum. The degree of contact rate of change can also be used to filter or smooth variation in signals resulting from cardiac cycle mechanical events.

The present invention also permits simultaneous measurements by multiple electrodes 12, 50, 52 on catheter 14. Signals having distinct frequencies or multiplexed in time can be generated for each electrode 12, 50, 52. In one constructed embodiment, for example, signals with frequencies varying by 200 Hz around a 20 kHz frequency were used to obtain simultaneous distinct measurements from multiple electrodes 12, 50, 52. Because the distinct frequencies permit differentiation of the signals from each electrode 12, 50, 52, measurements can be taken for multiple electrodes 12, 50, 52 simultaneously thereby significantly reducing the time required for mapping and/or EP measurement procedures. Microelectronics permits precise synthesis of a number of frequencies and at precise quadrature phase offsets necessary for a compact implementation of current sources and sense signal processors. The extraction of information in this manner from a plurality of transmitted frequencies is well known in the field of communications as quadrature demodulation. Alternatively, multiple measurements can be accomplished essentially simultaneously by multiplexing across a number of electrodes with a single frequency for intervals of time less than necessary for a significant change to occur.

Although several embodiments of this invention have been described above with a certain degree of particularity, those skilled in the art could make numerous alterations to the disclosed embodiments without departing from the scope of this invention.

All directional references (upper, lower, upward, downward, left, right, leftward, rightward, top, bottom, above, below, vertical, horizontal, clockwise and counterclockwise) are only used for identification purposes to aid the reader's understanding of the present invention, and do not create limitations, particularly as to the position, orientation, or use of the invention. Joinder references (attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not as limiting. Changes in detail or structure may be made without departing from the invention as defined in the appended claims.

What is claimed is:

1. A system for displaying contact between a catheter and a tissue in a body, comprising:
a catheter comprising a distal portion and a sensor; and
a controller in operable communication with the sensor and configured to receive as input therefrom a reading indicative of a degree of contact between the distal portion and the tissue, to calculate a degree of contact between the distal portion of the catheter and the tissue, and to provide an output indicative of the degree of contact.

2. The system of claim 1 wherein the sensor comprises at least one electrode.

3. The system of claim 1 wherein the sensor senses a first impedance component.

4. The system of claim 3 wherein the controller is configured to display the first impedance component and a second impedance component.

5. The system of claim 1 wherein the sensor is a force sensor.

6. The system of claim 1 wherein the sensor is an optical sensor.

7. The system of claim 1, further comprising a display coupled to the controller.

8. The system of claim 7, wherein the controller is configured to output a model of the tissue and a model of the catheter to be displayed on the display.

9. The system of claim 7 wherein the controller is configured to display a waveform indicative of the degree of contact on the display.

10. The system of claim 7 wherein the controller is configured to display a meter indicative of the degree of contact on the display.

11. The system of claim 10 wherein an appearance of the meter changes as the sensor reading changes.

12. The system of claim 10 wherein an appearance of the meter changes when the sensor reading crosses a programmable threshold value.

13. The system of claim 11 wherein the meter changes color as the sensor reading crosses a programmable threshold value.

14. The system of claim 8 wherein the controller is configured to display a beacon projected onto the displayed catheter model.

15. The system of claim 14 wherein, as the sensor reading varies, the beacon changes in a manner selected from the group consisting of color, size, length, intensity, shape and combinations thereof.

16. The system of claim 14 wherein the controller is configured to identify a portion of the sensor in contact with the tissue and to adjust the projected beacon to display an orientation of the catheter relative to the tissue.

17. A method for displaying contact between a catheter and a tissue in a body, comprising:
providing a catheter having a distal portion and a sensor on the distal portion;
providing a controller in communication with a display;
establishing communication between the sensor and the controller;
acquiring a sensor reading indicative of a degree of contact between the distal portion of the catheter and the tissue;
calculating a degree of contact between the sensor and the tissue; and
displaying a graphical representation of the calculated degree of contact on the display.

18. The method of claim 17 wherein the acquiring step includes measuring an impedance component of the tissue in contact with the distal portion of the catheter.

19. The method of claim 18 wherein said calculating step includes the substep of calculating a composite value for two components of a complex impedance.

20. The method of claim 17 further comprising displaying a model of the catheter on the display, wherein the step of displaying a graphical representation of the calculated degree of contact on the display comprises displaying a graphical representation of the calculated degree of contact on the model of the catheter.

21. An article of manufacture, comprising:
a catheter with a sensor; and
a computer storage medium having a computer program encoded thereon for determining a degree of contact between a sensor and tissue in a body, said computer program including computer code for:
calculating a degree of contact in response to a signal from the sensor; and
generating a visual indicator of the degree of contact to be displayed on a display device.

22. The article of claim 21, wherein the computer program further comprises computer code for determining a location of the catheter in relation to the tissue in the body.

23. The article of claim 22, wherein the computer program further comprises computer code for calculating a location of the tissue and for generating a representation of the tissue to be displayed on the display device.

24. The article of claim 23, wherein the representation is an electro anatomical map of a heart.

25. The article of claim 23, wherein the computer program utilizes the degree of contact in calculating the location of the tissue.

26. The article of claim 24, further comprising a low pass filter.

27. The method of claim 17 wherein the acquiring step includes measuring impedance of a component of the catheter which is indicative of a degree of contract between the distal portion of the catheter and the tissue.
